(19) United States
(12) Patent Application Publication
(10) Pub. No.: US 2023/0250221 A1
(43) Pub. Date: Aug. 10, 2023

(54) LIQUID MIXTURES OF PROPOXYLATED PARA-TOLUIDINES

(71) Applicant: SALTIGO GmbH, Leverkusen (DE)

(72) Inventor: Thomas Hoepfner, Langenfeld (DE)

(73) Assignee: SALTIGO GmbH, Leverkusen (DE)

(21) Appl. No.: 18/011,239

(22) PCT Filed: Jun. 24, 2021
(86) PCT No.: PCT/EP2021/067363

§ 371 Date: Dec. 19, 2022

(30) Foreign Application Priority Data
Jun. 25, 2020 (EP) 20182396.0

(51) Int. Cl. C08G 59/50; C07C 215/08; C08G 59/56

(52) U.S. Cl. CPC C08G 59/504; C07C 215/08; C08G 59/56; C08G 59/5033

(57) ABSTRACT

The invention relates to mixtures of propoxylated 4-toluidines containing two or more different di- or tri-propoxy lated or higher propoxylated p-toluidines in specific weight ratios, to methods for preparing them and to their use as polymerization accelerators or vulcanization accelerators or as hardener components in epoxy resins.

The invention relates to mixtures of propoxylated 4-toluidines (para-toluidines) comprising two or more different di- or tripropoxylated or more highly propoxylated p-toluidines in specific ratios, to processes for preparation thereof and to the use thereof as polymerization accelerator or as vulcanization accelerator or as hardener component for epoxy resins.

Crosslinked polymers may be produced by free-radical polymerization. This involves using unsaturated polyesters, for example. The polymerization process is also referred to as curing. The polymerization is initiated by what are called curing agents for this group of polymers. These are generally free-radical initiators, for example peroxides. The best-known and most widely used curing agent is dibenzoyl peroxide. This also often involves using polymerization accelerators that accelerate the polymerization process and have an advantageous effect on the curing process and/or the product properties of the polymer. Some polymerization accelerators may advantageously be incorporated into the polymer via further functional groups. Tertiary amines in the form of N,N-disubstituted toluidines are an important group of such polymerization accelerators—for reasons including their low volatility and their more advantageous toxicological profile, especially the group of the ethoxylated and propoxylated toluidines.

The individual compound N,N-bis(2-hydroxypropyl)-p-toluidine [N,N-bis(2-hydroxypropyl)-4-toluidine, N,N-dipropoxy-p-toluidine, 1,1′-(p-tolylimino)dipropan-2-ol, CAS RN 38668-48-3; diisopropanol-p-toluidine, N,N-di(2-hydroxypropyl)-p-toluidine], i.e. “dipropoxylated” para-toluidine, is known. The higher homologs thereof have to date been disclosed merely generically.

RU2063960A describes ethoxylated p-toluidines and specifically the preparation thereof from 4-toluidine and 3 to 4 mol of ethylene oxide per mole of 4-toluidine used at 80±5° C. without the addition of solvents and without the addition of catalysts. This affords liquid mixtures of ethoxylated 4-toluidines, the composition of which is not described any further. It can be inferred from the chemical yields that the average degree of ethoxylation (number of ethylene oxide units per 4-toluidine molecule) is between 2 and 2.5. Nothing is known about the distribution of the individual homologs.

EP 1650184 A1 describes generic homologs of N,N-bis(2-hydroxyalkyl)-p-toluidine that contain less than 0.2% by weight of alkoxylated 3-toluidines, based on alkoxylated 4-toluidines. Specifically disclosed is the preparation thereof from 4-toluidine containing less than 0.2% by weight of 3-toluidine, and 2.2 to 5 mol, preferably 2.3 to 4 mol, more preferably 2.3 to 3.5 mol and especially 2.5 to 2.6 mol of alkylene oxide per mole of 4-toluidine.

For example, the reaction of 4-toluidine containing less than 0.2% by weight of 3-toluidine, based on 4-toluidine, with 2.5 mol of ethylene oxide per mole of 4-toluidine used, without the addition of solvents and without the addition of catalysts at 120° C., afforded an ethoxylated 4-toluidine that no longer contains any 4-toluidine (detection limit: 100 ppm), and constituted a mixture of <0.1% N-hydroxyethyl-4-toluidine, 50.1% N,N-bis(hydroxyethyl)-4-toluidine and 43.7% N-oxyethyl-N-(hydroxyethyloxyethylene)-4-toluidine, 5.4% tetraoxyethyl-4-toluidine, 0.7% pentaoxethyl-4-toluidine and a trace of hexaaoxethyl-4-toluidine.

The reaction of 4-toluidine containing less than 0.2% by weight of 3-toluidine, based on 4-toluidine, with 2.58 mol of ethylene oxide per mole of 4-toluidine used, without the addition of solvents and with 30% sodium methoxide solution as catalyst, at 120° C., afforded an ethoxylated 4-toluidine which constituted mixtures of 47.4% N,N-bis(hydroxyethyl)-4-toluidine, 43.4% N-oxyethyl-N-(hydroxyethyloxyethyl)-4-toluidine and traces of more highly ethoxylated compounds. The document contains the description of further examples, but for which the distribution of the homologs is unknown.

Since the reaction of 4-toluidine with ethylene oxide proceeds virtually quantitatively, at least when a catalyst is used, it is possible to estimate on the basis of the chemical mass yields that the average degree of ethoxylation (the sum total of m and n in general formula (I) in the document) is close to that of the molar ratios between ethylene oxide and 4-toluidine.

N,N-Bis(hydroxyethyl)-4-toluidine (CAS RN 3077-12-1) is sold by LANXESS Deutschland GmbH/Saltigo GmbH as a black to yellow/brown liquid or solidified product for use as a hardener component for epoxy resins.

Likewise sold by LANXESS Deutschland GmbH/Saltigo GmbH is an “overethoxylated N,N-bis(hydroxyethyl)-4-toluidine” containing less than 0.2% by weight of ethoxylated 3-toluidine, named Accelerator PT25E/2, as a colorless to pale yellow-brownish viscous liquid for use as a hardener component for epoxy resins.

CN101200432 A discloses the simpler N,N-bis(hydroxypropyl)aniline and its higher homologs. These are obtained from the reaction of N,N-bis(hydroxypropyl)aniline in the presence of 3 to 3.6 mol of ethylene oxide per mole of aniline used at temperatures of 145 to 165° C. in the presence of catalysts such as alkali metal hydroxides or mixed metal cyanides.

N,N-Bis(2-hydroxypropyl)-p-toluidine (CAS RN 38668-48-3) is known and is sold by LANXESS Deutschland GmbH/Saltigo GmbH, among other suppliers, as a pale yellow solidified melt for use as a hardener component for epoxy resins.

According to the field of use and epoxy resin system, experience has shown that the ethoxylated anilines, the ethoxylated toluidines or N,N-bis(2-hydroxypropyl)-p-toluidine are more advantageous. However, N,N-bis(2-hydroxypropyl)-p-toluidine has the disadvantage of being in the form of a solidified melt at room temperature, and so N,N-bis(2-hydroxypropyl)-p-toluidine has to be melted before use by heating the container containing the product. It has been found that various isomers of N,N-bis(2-hydroxypropyl)-p-toluidine have different melting points. The effect of this was that, on partial melting of the product, the lower-melting isomers accumulated in the liquid phase and, after removal of the already liquefied fraction, the melting point of the remaining solid matter rose ever further. In this respect, this phenomenon is a kind of “unintentional melt refining”. If one wanted to avoid this phenomenon, it would be necessary to heat the N,N-bis(2-hydroxypropyl)-p-toluidine up to distinctly higher temperatures than, for example, N,N-bis(hydroxyethyl)-p-toluidine, which constitutes a performance disadvantage. Because the middle carbon atom in the propylene oxide used as reactant for preparation is asymmetric and two molecules of R- or S-propylene oxide react with one molecule of 4-toluidine, N,N-bis(2-hydroxypropyl)-p-toluidine takes the form of a mixture of different isomers, for example RR, SS, or meso. In addition, the epoxy ring of the 4-toluidine can be attacked and opened at the terminal CH₂ group, which is predominant, or at the middle CH group.

A. Zoltanski; et al., Current Applied Polymer Science, 2018, 2 (2), 89-93, The Structure of Propoxylated p-Toluidine, Used as a Polymerization Accelerator or in Unsaturated Polyester Resin Curing, elucidated the different structures that form in the reaction of 4-toluidine with two molecules of racemic propylene oxide.

In-house analyses of the differently melting fractions of N,N-bis(2-hydroxypropyl)-p-toluidine showed that, for example, the optically inactive meso isomer melts at higher temperature than the optically active isomers. This means that N,N-bis(2-hydroxypropyl)-p-toluidine has the technical disadvantage of taking the form of a complex mixture in the form of a solidified melt, meaning that it can only be introduced into the industrial application in a complex manner as a homogeneous mixture of the individual components.

The technical problem addressed was therefore that of providing a form of propoxylated 4-toluidine that does not have the disadvantage of N,N-bis(2-hydroxypropyl)-p-toluidine, but can be used at least just as well or better as a polymerization accelerator or as a vulcanization accelerator or as a hardener component for epoxy resins in the polymer systems in which N,N-bis(2-hydroxypropyl)-p-toluidine can be used.

This object was surprisingly achieved by the provision of mixtures comprising two or more different compounds of the general formula (I)

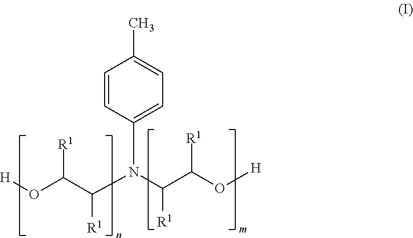

in which R¹ is hydrogen or methyl, but where the R¹ radicals on directly adjacent carbon atoms do not both represent hydrogen and do not both represent methyl, and m and n represent integers, characterized in that

4-toluidine is present in a proportion of not more than 2% by weight, preferably of 0.001% to 1% by weight, based on the total mass of all compounds of the formula (I) in the mixture, and

the compounds of the formula (I) in which the sum total of m and n is the integer 2 are present in the mixture in a proportion of not more than 20% by weight, preferably of 0.01% to 20% by weight, more preferably of 0.01% to 12% by weight, based on the total mass of all compounds of the formula (I), and

the compounds of the formula (I) in which the sum total of m and n is at least the integer 6 are present in the mixture in a proportion of not more than 40% by weight, preferably of 0.01% to 40% by weight, more preferably of 0.01% to 20% by weight, based on the total mass of all compounds of the formula (I).

The invention thus provides the mixtures of the invention comprising two or more different compounds of the formula (I). The wording “comprising two or more different compounds” rules out that exclusively one homolog such as N,N-bis(2-hydroxpropyl)-p-toluidine or N,N-bis(2-hydroxypropyloxypropylene)-p-toluidine is present. The compounds of the formula (I) in which the sum total of m and n is identical are each one homolog of N,N-bis(2-hydroxpropyl)-p-toluidine for the sum total of m and n. Homologs thus refer to compounds of the formula (I) in which the total number of oxypropylene units is different.

The corresponding distribution of the degree of propoxylation can be ascertained, for example, by GC-MS. By calibration of the GC evaluations with calibration substances, it is possible to make determinations of percentages by weight even by gas chromatography.

Preferably, the mixtures of the invention contain the compounds of the formula (I), in which the sum total of m and n is the integer 3, in the mixture in a proportion of 7% to 49% by weight, particularly preferably of 15% to 49% by weight, based on the total mass of all compounds of the formula (I).

Likewise preferably, the mixtures of the invention contain the compounds of the formula (I) in which the sum total of m and n is the integer 4 in the mixture in a proportion of 10% to 49% by weight, more preferably of 10% to 40% by weight, based on the total mass of all compounds of the formula (I).

In a further preferred embodiment, in the mixtures of the invention, every homologous group of compounds of the formula (I) is present in the mixture in a proportion of less than 50 percent by weight based on the total mass of all compounds of the formula (I) in the mixture. A homologous group of compounds of the formula (I) refers to any group of compounds having the same sum total of m and n, but which may differ in the combination of m and n. For example, the group of homologous compounds having the sum total of m and n=4 includes the compounds with
m=0 and n=4 or m=4 and n=0,
m=1 and n=3 or m=3 and n=1 and
m=n=2
and the individual isomers of the compounds having the abovementioned values of m and n.

This has the advantage that the substance mixture is classified as a polymer under chemical law in particular regions or countries and hence is subject to different conditions under chemical law as individual substances.

In a likewise preferred embodiment, the mixtures of the invention contain a proportion of 4-toluidine of less than 0.1% by weight based on the total mass of all compounds of the formula (I) in the mixture.

The mixtures of the invention may, as well the compounds of the formula (I), also contain further constituents. These may be residues of catalysts, water or other polymerization products of propylene oxide. The sum total of the percentages by weight of all compounds of the formula (I) and the percentages by weight of the further constituents add up to 100 percent by weight. Typically, the mixtures of the invention contain from 96% to 100% by weight of compounds of the formula (I).

The mixtures of the invention are typically in a liquid state of matter at room temperature and/or at temperatures of 5 to 40° C. In addition, the mixtures of the invention preferably do not include any solid constituents. The mixtures of the invention are preferably not suspensions. The mixtures of the invention can thus preferably be handled as homogeneous liquids at ambient temperature. This has the advantage that the mixtures of the invention, for use as a polymerization accelerator or as a vulcanization accelerator or as a hardener component, can be taken from the container in the liquid state in a simple manner, in exact amounts and in a stable and defined composition and can be used in the application in exact amounts and in a stable defined composition. According to the invention, the mixtures are in a liquid state of matter when they have a dynamic viscosity of 0.1 to 20 000 mPas (millipascal seconds) at 25° C.

Dynamic viscosity can be measured by different methods, for example by capillary or rotary viscometer. Unless stated otherwise, dynamic viscosities were measured to DIN 53019 with a rotary viscometer by the principle of the cone-plate measurement system (cf. DIN 53019-2, chapter 10.3) at the specified temperatures. The mixtures of the invention preferably have a dynamic viscosity of 500 to 20 000 mPas at a temperature of 25° C., measured to DIN 53019 with a rotary viscometer.

The mixtures of the invention are the products resulting directly from the production process of the invention.

The invention accordingly also provides the mixtures of the invention that are obtainable by the process of the invention.

The mixtures of the invention can surprisingly be produced by a simple and stable process. In the case of a detection limit by gas chromatography of 100 ppm, it is preferably no longer possible to detect any unconverted 4-toluidine in the mixtures of the invention. This is of major importance since the toluidines unalkylated on the nitrogen are classified as being severe hemotoxins and as being carcinogenic.

This process of the invention for preparing the mixtures comprises the reaction of the compounds of the formula (I) in which R¹ is hydrogen or methyl, but where the R¹ radicals on directly adjacent carbon atoms do not both represent hydrogen and do not both represent methyl, and in which m and n are the integer 1 (N,N-dipropoxy-p-toluidine), with from 1.0 to 4.0 mol, preferably from 1.25 to 2.50 mol, of propylene oxide (1,2-epoxypropane) per mole of 4-toluidine used in the presence of a catalyst.

The N,N-dipropoxy-p-toluidine used as reactant in the process of the invention, i.e. the compound of the formula (I) in which R¹ is hydrogen or methyl, but where the R¹ radicals on directly adjacent carbon atoms do not both represent hydrogen and do not both represent methyl, and in which m and n are the integer 1, may be a commercially acquired product, or a reactant obtained separately by in-house production with subsequent isolation, or a reactant obtained by in-house production without separate isolation. In the latter case, the reactant is produced in the reaction vessel in which the process of the invention is performed.

The process of the invention is preferably performed at temperatures of 80 to 150° C., preferably of 100 to 150° C. Lower temperatures may lead to incomplete conversions and in any case to uneconomically long reactions which, in the case of unadjusted metering rates of propylene oxide, entail a considerable and hence hazardous pressure buildup in the reactor. By contrast, higher temperatures—especially those above the limiting temperature Tₑₓₒ (according to TRAS 410) of 150° C. of N,N-dipropoxy-p-toluidine—can lead to uncontrolled exothermic breakdowns with a pressure buildup.

In the process of the invention, catalysts used are preferably alkali metal and alkaline earth metal hydroxides, alkali metal and alkaline earth metal carbonates, alkali metals, lithium alkyls, sodium hydride, complex hydrides such as lithium aluminum hydride, sodium bis(methoxyethoxy)aluminum dihydride, or alkali metal alkoxides. Catalysts used are more preferably alkali metal hydroxides, alkali metal and alkaline earth metal carbonates, most preferably sodium hydroxide or potassium hydroxide. For every mole of compound of the formula (I) in which R¹ is hydrogen or methyl, but where the R¹ radicals on directly adjacent carbon atoms do not both represent hydrogen and do not both represent methyl, and in which m and n are the integer 1 (N,N-dipropoxy-p-toluidine), preferably 0.01 to 0.05 mol, more preferably 0.02 to 0.035 mol, of catalyst is used in the process of the invention.

The N,N-dipropoxy-p-toluidine reactant, i.e. the compound of the formula (I) in which R¹ is hydrogen or methyl, but where the R¹ radicals on directly adjacent carbon atoms do not both represent hydrogen and do not both represent methyl, and in which m and n are the integer 1, for the process of the invention can be provided by reaction of 4-toluidine with from 1.8 to 2.2 mol, preferably from 1.9 to 2.1 mol, of propylene oxide per mole of 4-toluidine used, at temperatures of 80 to 150° C., preferably from 100 to 150° C., more preferably from 110 to 150° C., in the absence of catalysts.

This means that the process of the invention for preparing the mixtures of the invention also encompasses the reaction of 4-toluidine with from 1.8 to 2.2 mol, preferably from 1.9 to 2.1 mol, of propylene oxide per mole of 4-toluidine used, at temperatures of 80 to 150° C., preferably from 100 to 150° C., more preferably from 110 to 150° C., in the absence of catalysts for provision of the reactant, i.e. the compound of the formula (I), in which R¹ is hydrogen or methyl, but where the R¹ radicals on directly adjacent carbon atoms do not both represent hydrogen and do not both represent methyl, and in which m and n are the integer 1, with subsequent reaction of the reactant with from 1.0 to 4.0 mol, preferably from 1.25 to 2.50 mol, of propylene oxide per mole of 4-toluidine used in the presence of a catalyst.

The process of the invention is typically performed in such a way that, before commencement of the reaction, a temperature at which the component to be propoxylated, i.e. 4-toluidine or N,N-dipropoxy-p-toluidine, is in liquid form is chosen. Then the reactor is inertized with an inert gas, for example nitrogen, and the internal pressure is subsequently reduced to a pressure of about 50 to 700 hectopascal (hPa). In the inventive propoxylation of N,N-dipropoxy-p-toluidine, the catalyst is preferably added before the reactor is closed. The catalysts of the invention are preferably added in solid form, for example in the form of flakes, beads or powder. It is also possible to use it as an aqueous solution, in which case the water can be distilled out before further reaction or this removal of the water can be dispensed with.

Preferably, propylene oxide still present in the reactor after the reaction of 4-toluidine or N,N-dipropoxy-p-toluidine is driven out with inert gas.

Typically, the reaction of 4-toluidine or N,N-dipropoxy-p-toluidine with propylene oxide is conducted in a closed pressure-tight reactor, for example an autoclave. In the reaction, proceeding from the pressure established beforehand, rises in pressure in the range from 400 hPa to 2000 hPa are typically achieved. Absolute pressures in the reactor of more than 0.3 megapascal (MPa) are typically not achieved. However, these pressures may be distinctly exceeded when the gaseous propylene oxide is added in full to the 4-toluidine or N,N-dipropoxy-p-toluidine component to be propoxylated in the reactor either before or after attainment of the reaction temperature, or is metered into the reactor too quickly before or after attainment of the reaction temperature. Both should be avoided in practice for safety reasons. Typically, the propylene oxide is metered into the reaction mixture formed from the 4-toluidine or N,N-dipropoxy-p-toluidine and propylene oxide component to be propoxylated such that a pressure of about 0.2 MPa is not exceeded.

Propylene oxide is typically used in commercially available racemic form having a purity of at least 99%. It is also possible to use the individual enantiomers, i.e. R- and/or S-propylene oxide or any desired mixtures thereof, in the process of the invention.

The inventive reaction of N,N-dipropoxy-p-toluidine with propylene oxide is always conducted with catalyst. By contrast, the propoxylation of 4-toluidine to give N,N-dipropoxy-p-toluidine is conducted in the absence of catalysts.

Preference is given to conducting the process of the invention in the absence of solvents.

In a preferred embodiment of the process of the invention, the N,N-dipropoxy-p-toluidine reactant is provided by reacting a 4-toluidine having a proportion of not more than 0.5% by weight of 3-toluidine based on 4-toluidine. In an alternative preferred embodiment of the process of the invention, the N,N-dipropoxy-p-toluidine reactant is provided by reacting a 4-toluidine having a proportion of not more than 0.2% by weight of 3-toluidine based on 4-toluidine.

In order to obtain the specific, particularly pure 4-toluidine with a limited 3-toluidine content, it is necessary to remove the 3-toluidine from the technical grade 4-toluidine by careful distillation since the boiling points of the two isomers are close to one another (b.p. of 4-toluidine: 200.5° C.; b.p. of 3-toluidine: 203.4° C.).

A further means of preparing 4-toluidine with a maximum 3-toluidine content of 0.2% by weight is to recrystallize technical grade N-acetyl-4-toluidine, followed by hydrolysis and distillation. 4-Toluidine with a 3-toluidine content of less than 0.2% by weight is prepared in a preferred manner by first nitrating toluene, distillatively removing the two unwanted 2- and 3-nitrotoluene isomers from the resultant isomer mixture of 2-, 3- and 4-nitrotoluene by optimizing the reflux ratio very efficiently to give the required purity, and then subjecting the 4-nitrotoluene obtained to a hydrogenation to form 4-toluidine in the desired purity. In this way, it is possible to provide 4-toluidine having a content of less than 0.2% by weight, preferably of less than 0.1% by weight, of 3-toluidine even in industrial amounts.

The inventive mixtures cannot be prepared in one step proceeding from 4-toluidine in that either the total amount of propylene oxide required and the catalyst or the total amount of propylene oxide required without catalyst are added to the initial charge of 4-toluidine. In this case, unwanted by-products are formed to a distinct degree and/or the 4-toluidine used is converted incompletely.

In the performance of the preferred embodiments of the process of the invention, it is crucial that the narrow limits of the parameters with regard to the molar ratios between propylene oxide and 4-toluidine and/or between catalyst and 4-toluidine and/or the reaction temperature are observed in order to achieve the desired narrow distribution of the individual homologs of propoxylated 4-toluidine of the formula (I). Since these parameters influence one another, combination of all the above limits or all the below limits of the parameters in association with other parameters, for example the metering rate, the mixing of the liquid reaction phase of 4-toluidine, catalyst and gaseous propylene oxide metered in, can have the effect that a noninventive mixture of propoxylated 4-toluidines is formed in individual cases. However, the person skilled in the art, by adjusting these parameters within these tight limits, depending on the experimental construction or size of the reaction vessel, will be able to ascertain, without particular difficulty in a simple manner, the suitable combination of parameters which lead to the mixtures of the invention within the aforementioned limits.

The yield of the propoxylation of the invention is virtually quantitative and is limited only by losses in handling, as occur, for example, in the case of transfer through adhesion of residual amounts to the reactor wall. After the propoxylation has ended, it has been found to be useful to cool the reaction mixture down to a temperature in the range from 60 to 100° C. and to pass nitrogen through the reaction mixture for a certain period of time in order to remove any propylene oxide present completely from the system.

The reaction mixture can be worked up by methods known to the person skilled in the art or else used further directly.

The use of propylene oxide as propoxylated agent offers the advantage over other reagents (for example 1-chloropropan-2-ol, 1-bromopropan-2-ol or 1-iodopropan-2-ol) that there is no need to use auxiliary reagents (such as stoichiometric amounts of bases as hydrogen halide scavengers) and there is correspondingly no formation of salts that would have to be removed in a separate step. Another disadvantage of the use of halopropanols is, for example, corrosion on the metal apparatuses used.

The invention further provides for the use of the mixtures of the invention as polymerization or vulcanization accelerators, preferably in the polymerization of polyesters, especially of unsaturated polyesters, or as hardener component for epoxy resins. It has been found to be useful to use the mixture of the invention in an amount of 0.1-5% by weight. The polymerization in which the mixtures of the invention can be used is preferably a free-radical polymerization.

The mixtures of the invention, in the polymerization system in which the known N,N-dipropoxy-p-toluidine is used advantageously over ethoxylated or other alkylated 4-toluidines, have improved handling and/or better processibility and/or a lower required application rate and/or higher reactivity. The replacement of N,N-dipropoxy-p-toluidine, which is known to take the form of a solid melt, by the liquid mixtures of the invention in the free-radical polymerization of polyesters, especially of unsaturated polyesters, or as hardener component for epoxy resins can advantageously influence the physicochemical and/or physicomechanical properties of the polymers produced therewith.

In an alternative embodiment, it is a feature of the mixtures of the invention that contain a small proportion of propoxylated 3-toluidines that they can be used particularly advantageously as polymerization or vulcanization accelerator in the production of colorless polymers, since the use thereof does not lead to any discoloration of the polymer. Depending on the desired use of the polymer, this may be very important and may not be achievable with all polymerization and vulcanization accelerators of the prior art.

The invention also provides a polymeric product obtainable by polymerization, preferably of a polyester, especially of unsaturated polyester, in the presence of the mixtures of the invention as a polymerization or vulcanization accelerator or as a hardener component for epoxy resins. The polymerization in which the mixtures of the invention can be used is preferably a free-radical polymerization.

### EXAMPLES

### Examples 1a to 1e: Production of Propoxylated Toluidine Proceeding from N,N-dipropoxy-p-toluidine

### Example 1a

A 3 liter autoclave (stainless steel) with stirrer, internal thermometer, immersed introduction tube for the propylene oxide and riser tube for removal was initially charged with 1425 g of 98% N,N-dipropoxy-p-toluidine [compound of the formula (I) in which m and n are each the integer 1; 6.25 mol] in molten form and 10.9 g of about 90% potassium hydroxide flakes (0.175 mol). The autoclave was closed, inertized by injecting nitrogen, decompressed and evacuated to about 670 hectopascal (hPa) (absolute pressure). The contents were heated to >80° C. in order to largely melt the N,N-dipropoxy-p-toluidine. Then the melt was heated up to the desired reaction temperature (120° C.). At this temperature, the envisaged amount of propylene oxide (here 690.1 g=11.88 mol, corresponding to 1.9 molar equivalents based on N,N-dipropoxy-p-toluidine, i.e. a total of 3.9 molar equivalents based on 4-toluene) was metered in at a rate of about 163 g/h, with achievement of a pressure of 0.18 MPa (absolute pressure) for a short time. This pressure was typically not exceeded in the other examples either. About 90 min after the metered addition had ended, the total pressure had dropped to a pressure of about 800 hPa that was then constant for about 15 min. This was followed by stirring at reaction temperature for a further 60 min, then cooling to 40° C., compensating for the reduced pressure with nitrogen, blowing out any propylene oxide still present with nitrogen, and dispensing of the mixture through a clarifying filter. 2109.3 g (yield: 99.200 of the use amounts) of product was obtained, which has the following distribution (in percent by weight) of the homologs of the formula (I):

| | m = 1; | m = 1; | | | |
|---|---|---|---|---|---|
| | n = 1 | n = 2 | m + n = 4 | m + n = 5 | m + n > 5 |
| Content of | 0.51% by | 41.7% by | 35.6% by | 14.6% by | 6.27% by |
| compound of | weight | weight | weight | weight | weight |
| the formula | | | | | |
| (I) | | | | | |

Examples 1b to 1e were conducted analogously—if appropriate in a 0.5 liter autoclave. Rather than the amounts envisaged in example 1a, examples 1b to 1e were conducted with the data specified in table 1.

TABLE 1
Reaction data of examples 1b to 1e

| | Example 1b | | Example 1c | | Example 1d | | Example 1e | |
|---|---|---|---|---|---|---|---|---|
| Reaction data | inventive | | inventive | | inventive | | noninventive | |
| Autoclave size | 3 | L | 3 | L | 0.5 | L | 0.5 | L |
| 98% N,N- | 1425 | g | 1425 | g | 226 | g | 226 | g |
| dipropoxy-p- | 6.25 | mol | 6.25 | mol | 1.00 | mol | 1.00 | mol |
| toluidine | | | | | | | | |
| Catalyst: | 99.8% sodium | | 99.8% sodium | | 90% potassium | | 90% potassium | |
| | hydroxide | | hydroxide | | hydroxide | | hydroxide | |
| | beads | | beads | | flakes | | flakes | |
| Amount of | 5.73 | g | 5.73 | g | 1.13 | g | 1.13 | g |
| catalyst | 0.143 | mol | 0.143 | mol | 0.018 | mol | 0.018 | mol |
| | 2.3 | mol % ¹⁾ | 2.3 | mol % ¹⁾ | 1.8 | mol % ¹⁾ | 1.8 | mol % ¹⁾ |
| Reaction | 120° | C. | 120° | C. | 120° | C. | 120° | C. |
| temperature | | | | | | | | |
| Propylene | 671.9 | g | 635.6 | g | 58.2 | g | 29.1 | g |
| oxide | 11.56 | mol | 10.94 | mol | 1.00 | mol | 0.50 | mol |
| | 1.85 | moleq.²⁾ | 1.75 | moleq.²⁾ | 1.00 | moleq.²⁾ | 0.50 | moleq.²⁾ |
| Yield | 2047 | g | 2012 | g | 278 | g | 250 | g |
| | 97.4% of the | | 97.4% of the | | 97.4% of the | | 97.6% of the | |
| | amounts used | | amounts used | | amounts used | | amounts used | |
| Content of | | | | | | | | |
| compound of | | | | | | | | |
| the formula (I) | | | | | | | | |

| | | | | |
|---|---|---|---|---|
| m = 1; n = 1 | 0.95% by | 1.31% by | 11.0% by | 47.4% by |
| | weight | weight | weight | weight |
| m = 1; n = 2 | 44.6% by | 47.9% by | 72.2% by | 41.9% by |
| | weight | weight | weight | weight |
| m + n = 4 | 32.0% by | 31.0% by | 13.0% by | 10.5% by |
| | weight | weight | weight | weight |
| m + n = 5 | 14.0 | 12.4% by | 3.65% by | <0.1% by |
| | | weight | weight | weight |
| m + n > 5 | 7.66 | 6.20% by | <0.1% by | <0.1% by |
| | | weight | weight | weight |
| State of matter | liquid, | liquid, | liquid, | significantly |
| at 20° C. | slightly | slightly | viscous | viscous, |
| | viscous | viscous | | predominantly |
| | | | | with solid |
| | | | | fractions |
| State of matter | distinctly | distinctly | highly | solid with |
| at 5° C. | viscous | viscous | viscous | vitreously |
| | | | | solidified |
| | | | | fractions |
| State of matter | vitreously | vitreously | vitreously | solid with |
| at −15° C. | solidified | solidified | solidified | vitreously |
| | | | | solidified |
| | | | | fractions |

¹⁾ 1 mol %: 0.01 mol of base based on 1 mol of N,N-dipropoxy-p-toluidine used
²⁾x moleq.: x mol of propylene oxide based on 1 mol of N,N-dipropoxy-p-toluidine used

### Examples 2a to 2e: Production of Propoxylated Toluidine Proceeding from 4-toluidine

### Example 2a

A 3 liter autoclave (stainless steel) with stirrer, internal thermometer, immersed introduction tube for the propylene oxide and riser tube for removal was initially charged with 672.1 g of 99.7% 4-toluidine (6.25 mol) in molten form. The autoclave was closed, inertized by injecting nitrogen, decompressed and evacuated to about 670 hPa (abs.). The contents were heated up to >45° C., without stirring at first, in order to fully melt the 4-toluidine. Then the melt was heated further up to the desired reaction temperature (120° C.). At that temperature, 726.4 g of propylene oxide (12.49 mol) was metered in at a rate of about 163 g/h in the absence of catalysts, with attainment of a maximum pressure of 0.27 MPa (abs.) for a short period of time. This pressure was typically not exceeded in the other examples either. About 3.5 h after the metered addition had ended, the total pressure had dropped to a pressure of about 770 hPa that was then constant for about 15 min. Stirring was then continued at reaction temperature for a further 60 min, then the mixture was cooled to 80 to 100° C., and the reduced pressure was compensated for with nitrogen. By means of sampling, it is possible to verify whether the typical composition of N,N-dipropoxy-p-toluidine has been attained.

Subsequently, the envisaged amount (2.8 mol % based on 4-toluidine used) of solid about 90% potassium hydroxide was added, and the autoclave was closed again, inertized as described above, evacuated and heated up to the desired reaction temperature of 120° C. Subsequently, a further 690.1 g (11.88 mol) of propylene oxide was metered in at about 163 g.h, with observation of a pressure rise toward the end by about 760 hPa to about 0.143 MPa (absolute). About 50 min after the metered addition had ended, the total pressure had dropped to the original pressure of 670 hPa. This was followed by stirring at reaction temperature for a further 60 min, then cooling to 40° C., compensating for the reduced pressure with nitrogen, blowing out any propylene oxide still present with nitrogen, and dispensing of the mixture through a clarifying filter. 2087.4 g (yield: 99.4% of the amounts used) of product was obtained, which contains the following distribution (in percent by weight) of the homologs of the formula (I):

| | m = 1; | m = 1; | | | |
|---|---|---|---|---|---|
| | n = 1 | n = 2 | m + n = 4 | m + n = 5 | m + n > 5 |
| Content of | 1.69% by | 38.9% by | 35.2% by | 15.6% by | 7.00% by |
| compound of | weight | weight | weight | weight | weight |
| the formula | | | | | |
| (I) | | | | | |

Examples 2b to 2e were conducted analogously—if appropriate in a 0.5 liter autoclave. Rather than the amounts envisaged in example 2a, examples 2b to e were conducted with the data specified in table 2.

TABLE 2
Reaction data of examples 2b to 2e

| | Example 2b | | Example 2c | | Example 2d | | Example 2e | |
|---|---|---|---|---|---|---|---|---|
| Reaction data | inventive | | inventive | | inventive | | noninventive | |
| Autoclave size | 0.5 | L | 0.5 | L | 0.5 | L | 0.5 | L |
| 99.7% 4- | 107.5 | g | 107.5 | g | 107.5 | g | 77.4 | g |
| toluidine | 1 | mol | 1 | mol | 1 | mol | 0.72 | mol |

1st stage

| | | | | | | | | |
|---|---|---|---|---|---|---|---|---|
| Propylene | 116.2 | g | 116.2 | g | 116.2 | g | 83.6 | g |
| oxide | 2 | mol | 2 | mol | 2 | mol | 1.44 | mol |
| | 2.0 | moleq.²⁾ | 2.0 | moleq.²⁾ | 2.0 | moleq.²⁾ | 2.0 | moleq.²⁾ |
| Reaction | 110° | C. | 130° | C. | 120° | C. | 120° | C. |
| temperature | | | | | | | | |

2nd stage

| Catalyst: | 90% | | 90% | | 90% | | 90% | |
|---|---|---|---|---|---|---|---|---|
| | potassium | | potassium | | potassium | | potassium | |
| | hydroxide | | hydroxide | | hydroxide | | hydroxide | |
| | flakes | | flakes | | flakes | | flakes | |
| Amount of | 1.75 | g | 1.75 | g | 1.75 | g | 1.26 | g |
| catalyst | 0.028 | mol | 0.028 | mol | 0.028 | mol | 0.020 | mol |
| | 2.8 | mol % ¹⁾ | 2.8 | mol % ¹⁾ | 2.8 | mol % ¹⁾ | 2.8 | mol % ¹⁾ |
| Reaction | 110° | C. | 130° | C. | 120° | C. | 120° | C. |
| temperature | | | | | | | | |
| Propylene | 101.6 | g | 101.6 | g | 58.1 | g | 177.8 | g |
| oxide | 1.75 | mol | 1.75 | mol | 1.0 | mol | 3.06 | mol |
| | 1.75 | moleq.²⁾ | 1.75 | moleq.²⁾ | 1.0 | moleq.²⁾ | 4.25 | moleq.²⁾ |
| Yield | 317.5 | g | 317.9 | g | 266.3 | g | 316 | g |
| | 97.1% of the | | 97.1% of the | | 93.9% of the | | 92.9% of the | |
| | amounts used | | amounts used | | amounts used | | amounts used | |
| Content of | | | | | | | | |
| compound of the | | | | | | | | |
| formula (I) | | | | | | | | |

| | | | | |
|---|---|---|---|---|
| m = 1; n = 1 | 2.27% by | 1.08% by | 12.9% by | 0.55% by |
| | weight | weight | weight | weight |
| m = 1; n = 2 | 47.8% by | 33.5% by | 74.9% by | 5.36% by |
| | weight | weight | weight | weight |
| m + n = 4 | 32.7% by | 35.3% by | 8.71% by | 16.0% by |
| | weight | weight | weight | weight |
| m + n = 5 | 11.3% by | 18.2% by | 0.39% by | 20.8% by |
| | weight | weight | weight | weight |
| m + n > 5 | 3.79% by | 10.2% by | 0.20% by | 56.3% by |
| | weight | weight | weight | weight |
| State of matter | liquid, | liquid, | liquid, | liquid, |
| at 20° C. | slightly | slightly | viscous | slightly |
| | viscous | viscous | | viscous |
| State of matter | distinctly | distinctly | highly | distinctly |
| at 5° C. | viscous | viscous | viscous | viscous |
| State of matter | vitreously | vitreously | vitreously | vitreously |
| at −15° C. | solidified | solidified | solidified | solidified |

¹⁾ mol %.: 0.01 mol of base based on 1 mol of 4-toluidine used
²⁾x moleq.: x mol of propylene oxide based on 1 mol of 4-toluidine used

1. A mixture comprising two or more different compounds of the general formula (I) 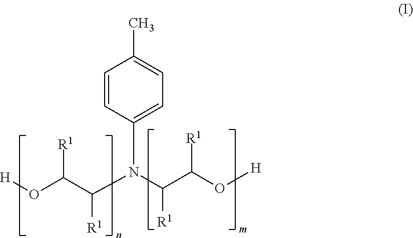
in which R¹ is hydrogen or methyl, but where the R¹ radicals on directly adjacent carbon atoms do not both represent hydrogen and do not both represent methyl, and m and n represent integers, wherein
4-toluidine is present in a proportion of not more than 2% by weight, based on the total mass of all compounds of the formula (I) in the mixture, and the compounds of the formula (I) in which the sum total of m and n is the integer 2 are present in the mixture in a proportion of not more than 20% by weight, based on the total mass of all compounds of the formula (I), and
the compounds of the formula (I) in which the sum total of m and n is at least the integer 6 are present in the mixture in a proportion of not more than 40% by weight, based on the total mass of all compounds of the formula (I).

2. The mixture as claimed in claim 1, wherein the compounds of the formula (I) in which the sum total of m and n is the integer 3 are present in the mixture in a proportion of 7% to 49% by weight, based on the total mass of all compounds of the formula (I).

3. The mixture as claimed in claim 1, wherein the compound of the formula (I) in which the sum total of m and n is the integer 4 is present in the mixture in a proportion of 10% to 49% by weight, based on the total mass of all compounds of the formula (I).

4. The mixture as claimed in claim 1, wherein every homologous group of compounds of the formula (I) is present in the mixture in a proportion of less than 50 percent by weight based on the total mass of all compounds of the formula (I) in the mixture.

5. The mixture as claimed in claim 1, wherein the mixture contains a proportion of 4-toluidine of less than 0.1% by weight based on the total mass of all compounds of the formula (I) in the mixture.

6. The mixture as claimed in claim 1, wherein the mixture is in a liquid state of matter at temperatures of 5 to 40° C.

7. The mixture as claimed in claim 1, wherein the mixture has a dynamic viscosity of 500 to 20 000 mPas at a temperature of 25° C., measured to DIN 53019 with a rotary viscometer.

8. The mixture as claimed in claim 1, wherein the mixture contains 96% to 100% by weight of compounds of the formula (I).

9. A process for preparing the mixture as claimed in claim 1, comprising the reacting the compounds of the formula (I) in which R¹ is hydrogen or methyl, but where the R¹ radicals on directly adjacent carbon atoms do not both represent hydrogen and do not both represent methyl, and in which m and n are the integer 1, with from 1.0 to 4.0 mol of propylene oxide per mole of 4-toluidine used in the presence of a catalyst.

10. The process for preparing the mixtures as claimed in claim 9, wherein the reacting step is effected at temperatures of 80 to 150° C.

11. The process for preparing the mixtures as claimed in claim 9, wherein the reacting step is effected in the presence of 0.01 to 0.05 mol of catalyst selected from the group consisting or alkali metal and alkaline earth metal hydroxides, alkali metals, lithium alkyls, sodium hydride, complex hydrides, lithium aluminum hydride, sodium bis(methoxyethoxy)aluminum dihydride, or alkali metal alkoxides, per mole of compound of the formula (I) used in which R¹ is hydrogen or methyl, but where the R¹ radicals on directly adjacent carbon atoms do not both represent hydrogen and do not both represent methyl, and in which m and n are the integer 1 (N,N-dipropoxy-p-toluidine).

12. The process for preparing the mixtures as claimed in claim 9, comprising the preparation of the compound of the formula (I) in which R¹ is hydrogen or methyl, but where the R¹ radicals on directly adjacent carbon atoms do not both represent hydrogen and do not both represent methyl, and in which m and n are the integer 1 (N,N-dipropoxy-p-toluidine), by reaction of 4-toluidine with from 1.8 to 2.2 mol of propylene oxide per mole of 4-toluidine used, at temperatures of 80 to 150° C. in the absence of catalysts.

13. The process for preparing the mixtures as claimed in claim 9, wherein the reacting step takes place in the absence of solvents.

14. The process for preparing the mixtures as claimed in claim 9, wherein the 4-toluidine has a proportion of not more than 0.2% by weight of 3-toluidine, based on 4-toluidine.

15. The use of the mixtures as claimed in claim 1 as polymerization or vulcanization accelerators, or as hardener component for epoxy resins.

16. A polymeric product obtainable by polymerization, of a polyester in the presence of the mixture as claimed in claim 1 as polymerization or vulcanization accelerator or as hardener component for epoxy resins.
